(19) United States
(12) Patent Application Publication
(10) Pub. No.: US 2006/0206182 A1
(43) Pub. Date: Sep. 14, 2006

(54) APPARATUS AND METHOD FOR DISPLACING TISSUE OBSTRUCTIONS

(76) Inventor: Stephen T. Pyles, Ocala, FL (US)

Correspondence Address: GARDNER GROFF SANTOS & GREENWALD, P.C., 2018 POWERS FERRY ROAD, SUITE 800, ATLANTA, GA, 30339, US

(21) Appl. No.: 11/382,844

(22) Filed: May 11, 2006

Related U.S. Application Data
(63) Continuation-in-part of application No. 11/158,309, filed on Jun. 21, 2005.
(60) Provisional application No. 60/640,648, filed on Dec. 30, 2004.
(60) Provisional application No. 60/581,531, filed on Jun. 21, 2004.

(51) Int. Cl. A61N 1/05; A61B 17/20; A61M 31/00; A61B 19/00

(52) U.S. Cl. 607/117; 606/129; 604/22; 604/500; 604/508; 607/118

(57) ABSTRACT

A catheter for displacing tissue obstructions. The shaft of the catheter has first and second conduits extending therethrough. Preferably, the catheter has a distensible, cuffed balloon positioned around the distal end of its shaft, in fluid communication with the first conduit. The catheter preferably also has a fluid delivery port located at its distal end for discharging fluid from the second conduit. Preferably, the catheter also includes a stylet for guiding the catheter into and through the epidural space.

### CROSS-REFERENCE TO RELATED APPLICATIONS

This application is a continuation-in-part of U.S. patent application Ser. No. 11/158,309, filed Jun. 21, 2005, which claims priority to both U.S. Provisional Patent Application Ser. No. 60/581,531, filed Jun. 21, 2004, and to U.S. Provisional Patent Application Ser. No. 60/640,648, filed Dec. 30, 2004. U.S. patent application Ser. No. 11/158,309 and Provisional Patent Application Ser. Nos. 60/581,531 and 60/640,648 are hereby incorporated herein by reference in their entireties for all purposes.

### TECHNICAL FIELD

The present invention relates generally to the field of surgical methods and devices, and more particularly to an apparatus and method for displacing tissue obstructions.

### BACKGROUND OF THE INVENTION

Lumbar epidural adhesions often occur as a result of a person undergoing back surgery. The adhesions, or scar tissue, tend to form around nerves and nerve roots. As a result, the adhesions apply pressure on the nerves and nerve roots, which in turn causes the person to feel pain in his or her back or legs.

To remove an adhesion, a physician often uses an MRI (i.e., magnetic resonance imaging) or an epidurogram to locate the general area of the adhesion. Once the area of the adhesion is known, the physician then inserts a catheter into the area of the adhesion. Next, the physician injects fluid, such as a saline mixture, into the adhesion so as to “break up” or displace or separate tissues. However, the use of fluids alone does not always provide sufficient results, as the adhesions may not break apart.

Spinal cord stimulation alleviates chronic pain resulting from multiple factors including adhesions or scar tissue applying pressure to the nerves and nerve roots by stimulating the central nervous system. The presence of such adhesions, fat, veins, and connective tissue membranes interfere with the accurate placement of leads for spinal cord stimulation. Often, the leads are inserted through a needle and placed in the epidural space, in close proximity to the spinal cord. However, accurate placement of the leads can be difficult because the practitioner must navigate through or around such adhesions, fat, veins, and connective tissue membranes.

Therefore, a need exists for an apparatus and method which would allow for greater ease in placing electrodes in the epidural space. Additional needs exist for an apparatus and method which could also be used to break apart adhesions and other regions in the epidural space (e.g., in the lower lumbar region often occurring following lumbar surgery).

### SUMMARY OF THE INVENTION

In example forms, the apparatus and method of the present invention provides a catheter for more effectively displacing tissue obstructions, such as lumbar epidural adhesions, fibrous connective tissue membranes, fat, and veins, when implanting spinal cord stimulator leads, such as percutaneous epidural leads or surgical leads, in the epidural space. The apparatus includes a catheter having a cuffed balloon for displacing obstructions and a fluid delivery port for injecting a saline solution to also displace the obstructions. Additionally, the catheter has a shape similar to that of the percutaneous epidural lead. The catheter's similarity in shape to that of a percutaneous epidural lead can create an adequate channel for the percutaneous epidural lead.

The catheter includes a shaft having first conduit and second conduits extending therethrough. Preferably, the catheter has a distensible balloon positioned around its distal end and in fluid communication with the first conduit, and a fluid delivery port also located at its distal end for discharging fluid from the second conduit. Preferably, the catheter further includes a malleable stylet for guiding the catheter through the epidural space. Also preferably, the balloon is a cuffed balloon.

In another aspect, the present invention provides a method for displacing tissue obstructions or lumbar adhesions including the steps of performing a laminectomy and inserting a catheter having a distensible balloon around its distal end and a fluid delivery port at a distal end thereof through the laminectomy; and inflating and deflating the balloon when the catheter encounters a tissue obstruction. Optionally, the method further includes the step of injecting a fluid comprising saline, corticosteroid, and/or hyaluronidase or other substances into the area of the obstruction through the fluid delivery port and then inflating and deflating the balloon to break up the adhesion.

In yet another aspect, the present invention provides a kit for performing percutaneous lysis of lumbar epidural adhesions. The kit includes a needle, a sterile drape, fluid couplings, a spinal cord stimulator lead, and a catheter having a cuffed balloon around its distal end and having a shape similar to that of the spinal cord stimulator lead, wherein all of the above are packaged in a single kit.

In still another aspect, the present invention provides a method of implanting a spinal cord stimulator lead in the spinal epidural space. The method includes the steps of performing percutaneous lysis of the epidural space using a catheter having a distensible, cuffed balloon and a fluid delivery conduit and inserting a lead comprising at least one electrode into the epidural space along a path cleared by the catheter.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a catheter in accordance with an example embodiment of the present invention.

FIG. 2 is a view of the distal or front end of the catheter of FIG. 1.

FIG. 3 is a cross-sectional view of the distal end of the catheter of FIG. 1.

FIG. 4 is a perspective view of a catheter in accordance with another example embodiment of the present invention.

FIG. 5 is a view of the distal or front end of the catheter of FIG. 4.

FIG. 6 is a cross-sectional view of the distal end of the catheter of FIG. 4.

FIG. 7 is a perspective view of a catheter in accordance with yet another example embodiment of the present invention.

FIGS. 8A and 8B present a flowchart representation of a method for performing percutaneous lysis of lumbar epidural adhesions using the catheter of FIGS. 1, 4, or 7.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

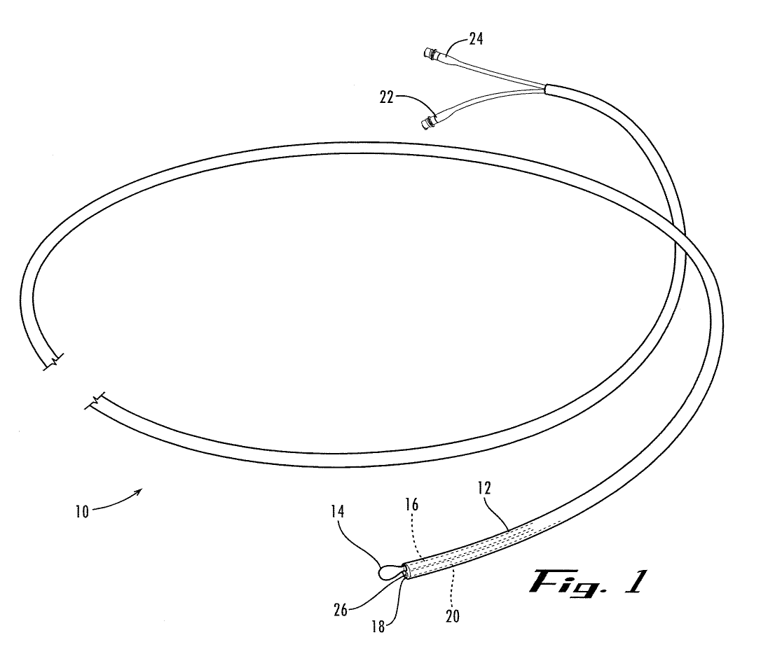

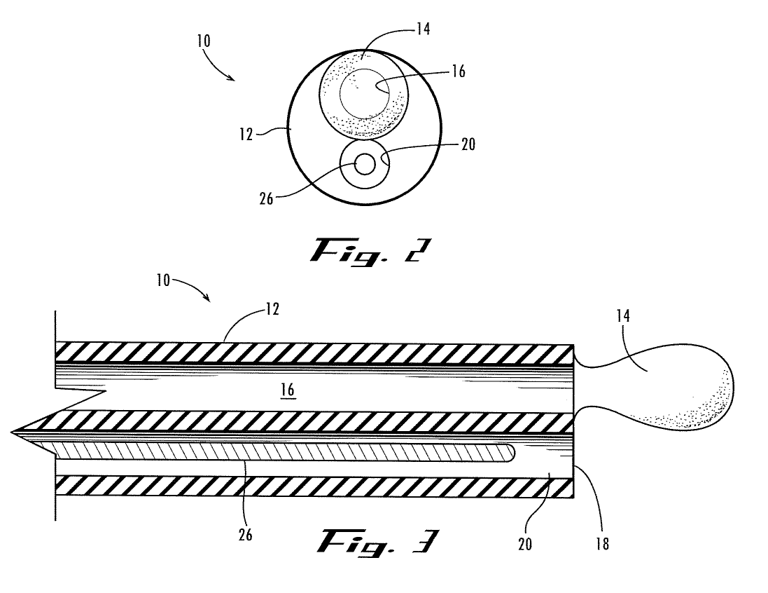

A catheter 10 for displacing tissue obstructions is described herein by way of an example embodiment shown in FIGS. 1-3. The catheter 10 has a biocompatible body or shaft 12 and a balloon 14 positioned at its distal end for displacing tissue obstructions, which can include lumbar epidural adhesions, connective tissue membranes, fats and veins. The balloon 14 is in fluid communication with a first lumen or conduit 16 extending through the catheter. The first lumen 16 carries a fluid, such as a sterilized liquid or air, under sufficient pressure to inflate and deflate the balloon 14. Preferably, the balloon 14 is constructed of a durable, yet distensible, material such as latex, although the present invention also contemplates the use of other distensible, biocompatible materials. The practitioner can alternately inflate and deflate the balloon 14 by selective delivery of pressurized fluid to displace tissues that prevent the passage or placement of the spinal cord stimulator lead.

The body 12 of the catheter 10 is preferably constructed of a biocompatible and somewhat flexible material, such as silicone, polyurethane, or polyethylene. It will be understood by those skilled in the art that various other biocompatible materials can be employed as well for the body 12.

A fluid injection port 18 is located at the distal end of the catheter 10. The port 18 delivers fluid from a second lumen or conduit 20 that extends through the body 12 of the catheter 10. The second lumen 20 carries fluid for directly injecting into the area of the adhesion. For example, a mixture of saline, corticosteroid, and hyaluronidase can be injected via the second lumen 20 and the port 18 to the site of the adhesion to displace an adhesion and/or reduce the inflammation. Preferably, the volume of the mixture is not more than about 20 milliliters. Also preferably, the amount of the hyaluronidase is limited to about 150 units to no more than about 1500 units, while the amount of the corticosteroid administered depends on the type of corticosteroid used. Those skilled in the art will understand how to determine the amount of corticosteroid to administer.

Preferably, inlet ports of the lumens 16 and 20 at the proximal end of the catheter 10 are each connected to their respective fluid sources with releasable, fluid-tight connectors 22 and 24, such as male “leur-lock” type connectors that couple with female leur-lock connectors of a fluid source(s), although other types of connectors for coupling the conduits of the catheter with a fluid source are within the scope of the present invention.

In the depicted embodiment of FIGS. 1-3, the lumens 16 and 20 are shown having diameters that are substantially similar. However, those skilled in the art will understand that the lumens 16 and 20 can have different diameters. For example, the diameter of the lumen 20 for delivering fluid directly to the site of the obstruction can be larger than the diameter of the lumen 16 for carrying fluid to the balloon, or vice versa. Those skilled in the art will also understand that one or both of the lumens 16 and 20 can extend along the outer body of the shaft 12 or within the shaft 12.

The catheter 10 also preferably includes a stylet 26 positionable within the second fluid conduit 20. Preferably, the stylet 26 is a slender and substantially rigid, but malleable, surgical wire for guiding the catheter 10 into and through the soft tissue. Such use of surgical wire allows the practitioner to view the location of the stylet with conventional imaging technology. The stylet 26 can be straight, or the stylet can have a tip that is angled or curved at an angle of about 30° to about 45°, for example, to improve steerablilty and control. In such instances where the tip of the stylet 26 is angled, the shape of the tip of the catheter 10 would typically conform to shape of the angled tip of the stylet. Preferably, the stylet 26 is removable from the catheter 10 such that once the catheter encounters an obstruction, the stylet can be removed and the second lumen 20 can be fitted with a connector, such as a male leur-lock connector, and coupled to a fluid source for delivering fluid directly to the area of the obstruction. Alternatively, the stylet 26 can extend through a third lumen of the catheter 10 such that the second fluid conduit 20 can be used for fluid injection while simultaneously guiding the catheter with the stylet.

Preferably, the size and shape of the catheter 10 is substantially similar to (or slightly larger or longer than) the size and shape of a conventional and commercially available percutaneous epidural lead for spinal cord stimulation, so as to create a suitable path through which the lead can be implanted. Thus, the body 12 of the catheter 10 is preferably generally cylindrical with a diameter of from about 0.6 mm to about 1.8 mm, as conventional percutaneous spinal cord stimulator leads are generally cylindrical and have a diameter of about 0.8 mm to about 1.5 mm and a length of about 30 cm to about 60 cm long, or even longer. Also preferably, the catheter has a cross-sectional geometry substantially similar to that of a cross-sectional geometry of the percutaneous spinal cord stimulator lead. However, it will be understood by those skilled in the art that various sizes and shapes can be employed without deviating from the scope of the present invention.

Optionally, the catheter 10 can include a marker, such as a radiographic strip or band near the tip of the catheter. The marker can aid the practitioner in guiding the catheter 10 under fluoroscopy or other conventional imaging technique into a proper placement in the epidural space.

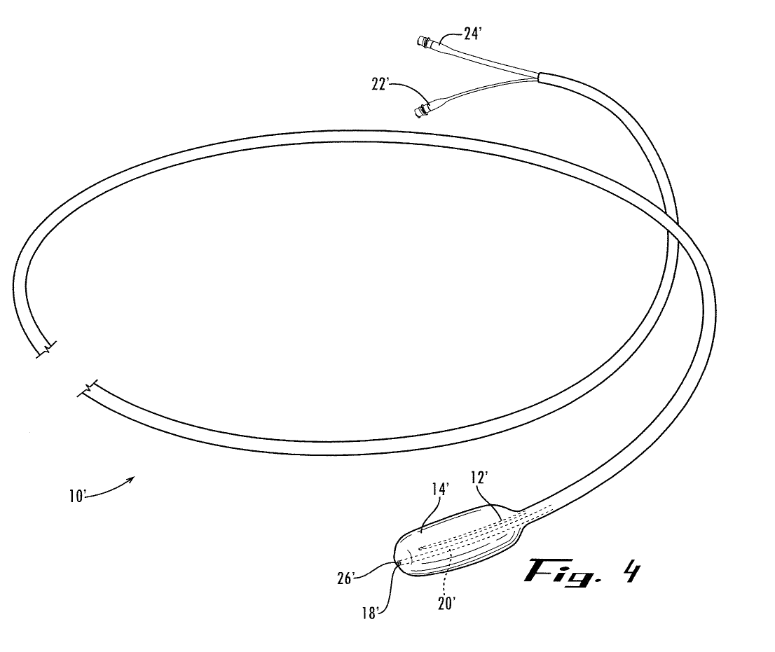

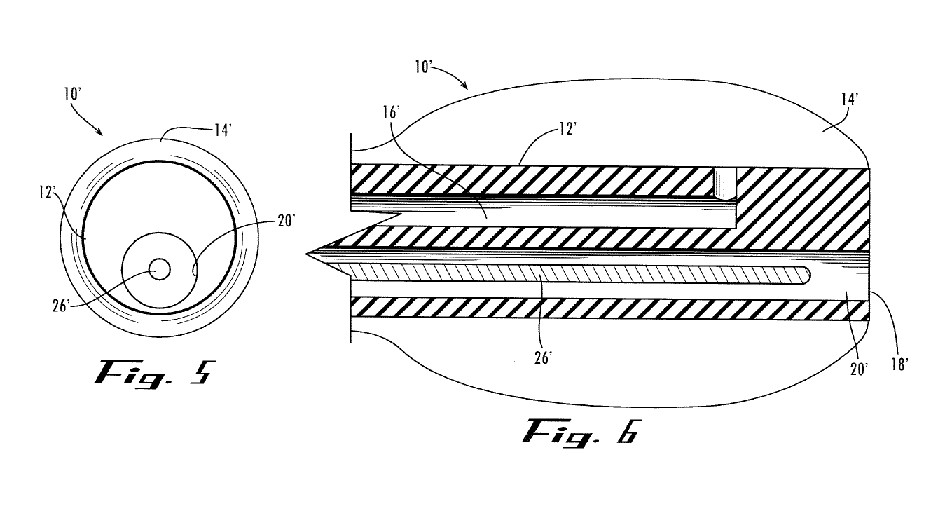

Another example embodiment of the catheter 10′ is shown in FIGS. 4-6. The catheter 10′ is substantially similar to the catheter 10, but with the exceptions noted herein. The catheter 10′ includes a cuffed balloon 14′ located around a distal end thereof. The depicted embodiment of FIGS. 4-6 shows that the balloon 14′ extends to the tip of the catheter 10′; however, those skilled in the art will understand that the balloon 14′ may not extend all the way to the tip. Preferably, the cuffed balloon 14′ has a generally oblong shape in the sense that the balloon is longer than it is wide when the balloon is inflated. In an example embodiment, the balloon 14′ extends from or near the tip of the shaft or body 12′. The cuffed balloon 14′ is in fluid communication with the conduit 16′ such that the balloon can expand generally radially outwardly about all or a portion of the circumference of the shaft or body 12′ at the distal end of the catheter 10′. Preferably, the balloon 14′ can be expanded to a size of about four to six times greater than the diameter of the body 12′ of the catheter 10′. Thus, preferably, the length of the balloon 14′ is at least, and more preferably, greater than four to six times greater than the diameter of the body 12′.

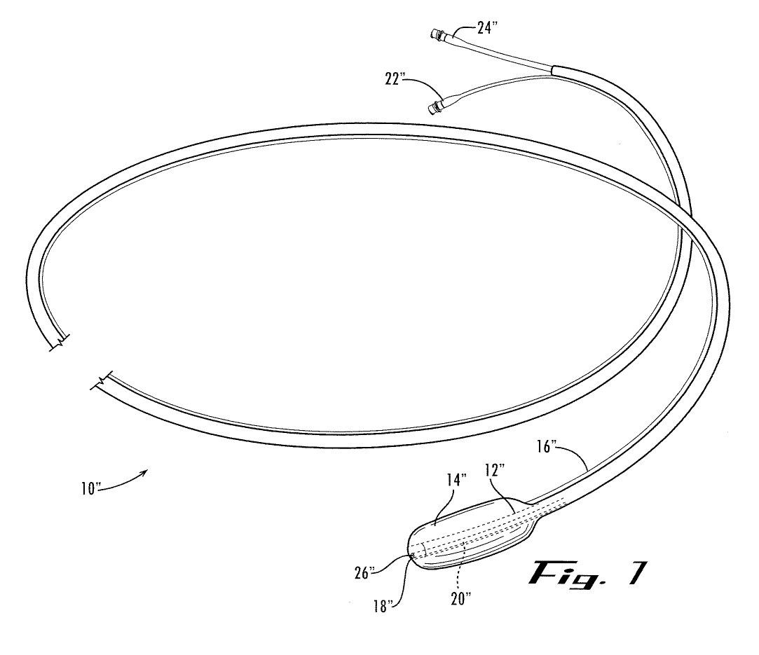

The lumen 16′ carries a fluid, such as a sterilized liquid or air, under sufficient pressure to inflate and deflate the balloon 14′. The diameter of the lumen 16′ for delivering a fluid to inflate the balloon 14′ is preferably smaller than the diameter of the lumen 20′ for carrying a fluid directly to the site of the obstruction. However, those skilled in the art will understand that the lumens 16′ and 20′ can have substantially the same diameter, or the diameter of the lumen 20′ for delivering fluid directly to the site of the obstruction can be smaller than the lumen 16′ for carrying fluid to the balloon. Those skilled in the art will understand how to determine the diameters of the lumens 16 and 20. Those skilled in the art will also understand that one or both of the lumens 16′ and 20′ can extend along the outer body of the shaft 12′, as shown in FIG. 7, or within the shaft 12′, and lumen 20′ may also serve as the lumen for the stylet 26′.

Preferably, the balloon 14′ is constructed of a durable, yet distensible, material such as latex, although the present invention also contemplates the use of other distensible, biocompatible materials. The practitioner can alternately inflate and deflate the balloon 14′ to laterally displace tissues that prevent the passage or placement of the spinal cord stimulator.

FIG. 7 is a perspective view of a catheter 10″ according to another example embodiment of the present invention. The catheter 10″ is substantially similar to the catheter 10′, but with the exceptions noted herein. The lumen 16″ is external to the shaft 12″ and has a diameter smaller than the diameter of the shaft 12″. Moreover, those skilled in the art further understand that both of the lumens 16″ and 20″ can extend along the outer body of the shaft 12″ or within the shaft 12″.

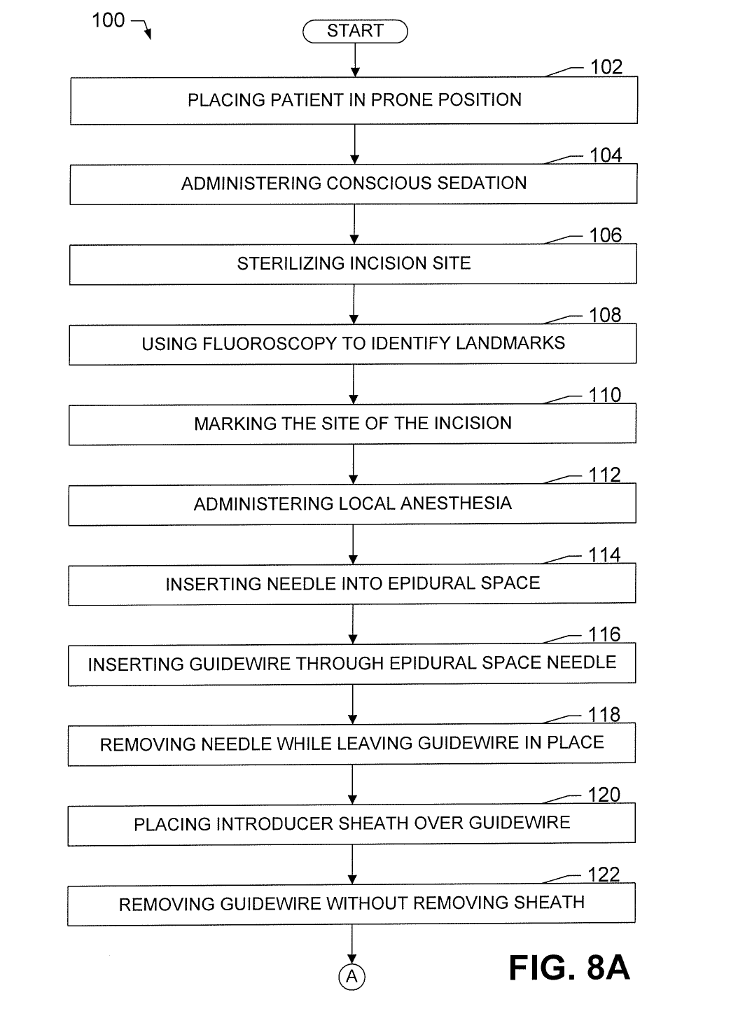

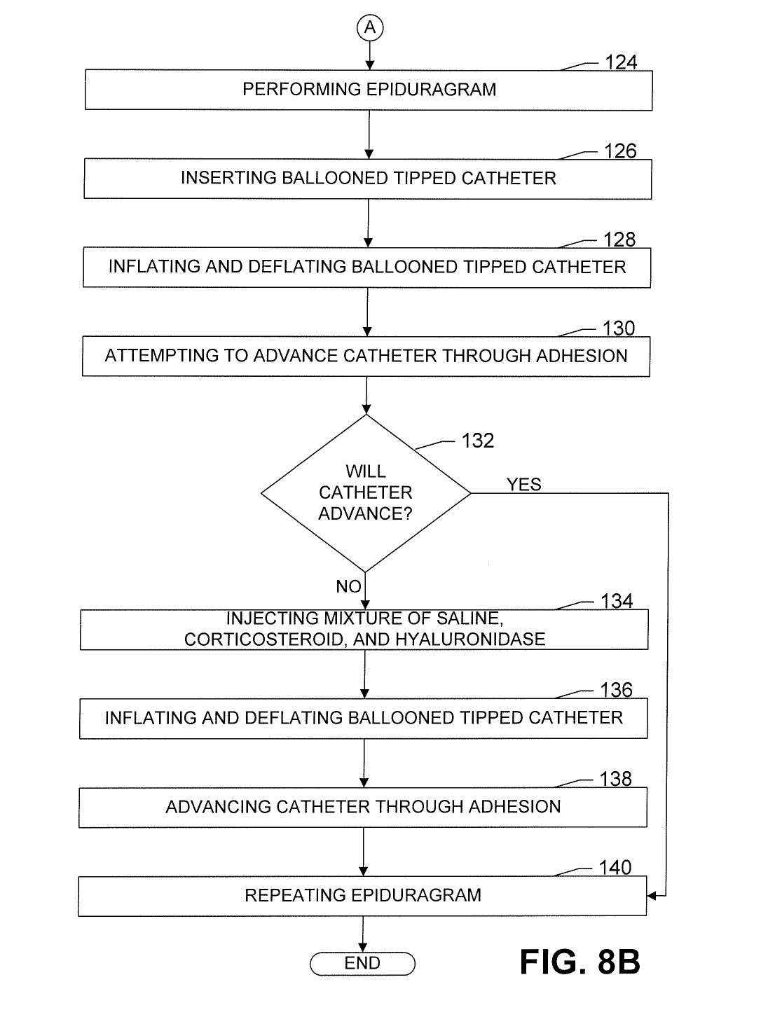

In one application, a method 100 for performing percutaneous lysis of lumbar epidural adhesions using the catheter 10, 10′, or 10″ is described herein by way of an example embodiment. With reference to FIGS. 8A and 8B, beginning at step 102, the practitioner places the patient in a prone position, and at step 104, administers conscious sedation to the patient using for example, opioid and benzodiazepine.

Next, at step 106, the practitioner sterilizes the area where the skin surface is to be punctured. For example, the practitioner can apply an antiseptic solution to the skin surface and then cover it with a sterile drape having an opening therein for access to the site to be punctured.

Then, at step 108, the practitioner uses fluoroscopy to identify landmarks. For example, the practitioner can use fluoroscopy to identify an oblique view of the vertebral column at L3 and the superior anterior lateral surface. At step 110, the practitioner marks the skin surface with a skin marker at the corresponding location.

At step 112, the practitioner applies a local anesthetic, such as for example 0.5% marcaine with epinephrine, to the skin and the soft tissues. Then, at step 114, the practitioner inserts a needle through the skin and into the epidural space. The practitioner can confirm the placement of the needle by injecting approximately two milliliters of a water-soluble contrast material through the needle and by viewing the resulting fluoroscopic image.

Next, the practitioner inserts a guidewire through the needle and into the epidural space at step 116. At step 118, the practitioner removes the needle from the epidural space while leaving the guidewire in the epidural space. The practitioner then places an introducer sheath over the guidewire at step 120, and removes the guidewire from the epidural space at step 122, while leaving the introducer sheath in place.

Next, the practitioner performs an epiduragram at step 124 by using approximately five milliliters of water-soluble contrast material, and preferably makes an x-ray copy of the image produced by the epiduragram.

Then at step 126, the practitioner inserts the balloon tipped catheter 10, 10′, or 10″, which preferably includes the stylet 26, 26′, and 26″, into the sheath and advances it into the region of the lumbar epidural adhesions. Once positioned, the practitioner inflates and deflates the balloon 14, 14′ or 14″ several times at step 128. After each inflation/deflation, the practitioner attempts to advance the catheter 10, 10′, or 10″ through the area of the adhesion at step 130. If, at step 132, the practitioner determines that the catheter 10, 10′, or 10″ will not advance, then at step 134, the practitioner injects a mixture of saline, corticosteroid, and hyaluronidase through the second conduit 20, 20′, or 20″ and the port 18, 18′, or 18″ and into the site of the adhesion to reduce the inflammation. Preferably, the volume of the mixture is not more than about 20 milliliters. Also preferably, the amount of the corticosteroid administered is limited to no more than about 80 milligrams, and the amount of the hyaluronidase is limited to no more than about 1500 units.

Alternatively, the practitioner can insert the catheter 10, 10′, or 10″ without the use of a sheath by inserting a guidewire or stylet 26, 26′, or 26″ through the second conduit 20, 20′, or 20″ and using the stylet to steer the catheter into the epidural space directly through the needle previously confirmed to be in the epidural space.

After the mixture is injected, the practitioner repeats a series of inflations and deflations of the balloon 14, 14′, or 14″ at step 136 and attempts to successfully advance the catheter 10, 10′, or 10″ at step 138. Then, at step 140, the practitioner repeats the epiduragram with approximately five milliliters of water-soluble contrast material after the termination of the procedure. This second epiduragram documents the lysis of the adhesions by demonstrating the resolution of the filling effects.

In another application, the practitioner can use the catheter 10, 10′, or 10″ to clear a path in the epidural space for placement of a spinal cord stimulator lead. For example, the practitioner can perform a laminectomy and can insert the catheter 10, 10′, or 10″ through a laminectomy. The practitioner can use the catheter 10, 10′, or 10″ to perform lysis of the epidural space, or in other words to break up the fat, veins, adhesions, and/or connective tissue membranes which would interfere with the placement of the spinal cord stimulator lead. The practitioner can inflate and deflate the balloon 14, 14′, or 14″ in an effort to displace the fat, veins, adhesions, and/or connective tissue membranes. If the practitioner cannot clear a suitable path through inflating and deflating the balloon 14, 14′, or 14″ the practitioner can inject a saline solution, such as one including corticosteroid and hyaluronidase, and then re-inflate and deflate the balloon 14, 14′, or 14″ to help displace the tissues. Once a suitable path has been cleared, the catheter 10, 10′, or 10″ is removed. A spinal cord stimulator lead comprising at least one electrode, and having a size and shape generally corresponding to that of the path cleared by the catheter 10, 10′, or 10″ is inserted into the epidural space along the path cleared by the catheter. The lead is then secured in place with sutures.

Optionally, the tools that the practitioner uses to insert and guide the catheter 10, 10′, or 10″ into the patient can be assembled into a single kit. For example, the kit can include one or more of a needle and/or scalpel, a catheter 10, 10′, or 10″ a sterile drape, fluid couplings, suturing supplies, a guidewire or stylet, a needle, an introducer sheath, and a spinal cord stimulator lead, the lead preferably generally matching the size and geometry of the catheter. In an example embodiment, the spinal cord stimulator lead is a percutaneous epidural lead. In an alternative embodiment, the spinal cord stimulator lead can be a surgical lead.

While the invention has been described with reference to example embodiments, it will be understood by those skilled in the art that a variety of modifications, additions and deletions are within the scope of the invention, as defined by the following claims.

What is claimed is:

1. A catheter for displacing tissue obstructions, comprising:
a shaft having first and second conduits extending therethrough;
a distensible balloon positioned around a distal end of the shaft and in fluid communication with the first conduit; and
a fluid delivery port located at the distal end of the catheter for discharging fluid from the second conduit.

2. The catheter of claim 1, further comprising a stylet for guiding the catheter into and through the epidural space.

3. The catheter of claim 2, wherein the stylet is positioned within the second conduit.

4. The catheter of claim 1, wherein the first conduit carries a sterilized fluid under sufficient pressure to expand the balloon.

5. The catheter of claim 1, wherein the balloon is a cuffed balloon that expands radially outwardly from at least a portion of the shaft's distal end.

6. The catheter of claim 1, wherein the second conduit is connectable to a fluid delivery source for delivery of a mixture of saline, a corticosteroid, and hyaluronidase.

7. In combination, the catheter of claim 1 and a percutaneous epidural lead, wherein the catheter has a cross-sectional geometry substantially similar to that of a cross-sectional geometry of the percutaneous epidural lead.

8. The catheter of claim 1, wherein the balloon is formed of latex.

9. The catheter of claim 1, further comprising a leur-lock connector for coupling one of said conduits to a fluid source.

10. A method of displacing tissue obstructions, said method comprising:
performing a laminectomy;
inserting a shaft of a catheter having a distensible balloon around a distal end of thereof and a fluid delivery port at a distal end thereof through the laminectomy; and
inflating and deflating the balloon when the catheter encounters a tissue obstruction.

11. The method of claim 10, further comprising the step of injecting a fluid comprising saline, corticosteroid, and/or hyaluronidase into the area of the obstruction through the fluid delivery port.

12. The method of claim 11, further comprising repeating the step of inflating and deflating of the balloon after injecting the fluid.

13. The method of claim 10, further comprising the step of using fluoroscopy to guide the catheter through the epidural space.

14. The method of claim 10, further comprising placement of a spinal cord stimulator lead within a path cleared by the displacement of tissue obstructions.

15. A kit for performing lysis of lumbar epidural adhesions, comprising:
a needle;
a sterile drape;
at least one fluid coupling;
a spinal cord stimulator lead; and
a catheter having a cuffed balloon around a distal end thereof, wherein the catheter has a shape similar to that of the spinal cord stimulator lead and wherein all of the above are packaged in a single kit.

16. The kit of claim 15, wherein the spinal cord stimulator lead is a percutaneous epidural lead.

17. A method of clearing a path for implanting a spinal cord stimulator lead in the spinal epidural space, said method comprising:
performing lysis of the epidural space using a catheter having a distensible, cuffed balloon and a fluid delivery conduit.

18. The method of claim 16, further comprising inserting a lead comprising at least one electrode into the epidural space along a path cleared by the catheter.
